(19) United States
(12) Patent Application Publication
(10) Pub. No.: US 2003/0077648 A1
(43) Pub. Date: Apr. 24, 2003

(54) Converting human DNA sequence data to computer-generated art imagery

(76) Inventor: George John Zelechowski, Cheektowaga, NY (US)

Correspondence Address: George John Zelechowski, 13 Mary Lou Lane, Depew, NY, 14043, US

(21) Appl. No.: 10/274,869

(22) Filed: Oct. 19, 2002

Related U.S. Application Data
(60) Provisional application No. 60/347,972, filed on Oct. 20, 2001.

(51) Int. Cl. C12Q 1/68; G06F 19/00; G01N 33/48; G01N 33/50

(52) U.S. Cl. 435/6; 702/20

(57) ABSTRACT

A procedure to convert human DNA sequence data subsets to computer-generated art image will allow visualization of a unique identifying sequence of DNA base-pairs based upon the Federal Bureau of Investigation open CODIS-compatible sample analysis methodology. This image will be absolutely unique to the individual and will never be repeated unless the DNA sampled is either from an identical twin sibling or from a genetically cloned human being. Also, it will be the exact same image produced from a single individual's DNA sample no matter what point in time during the existence of the individual that the sample is taken. The sole purpose of this new invention will be to create a unique work of art algorithmically based strictly on an individual subject's DNA sample. Its purpose is not designed nor intended to facilitate efficient, simple, and quick comparison of two different DNA samples as the standard charts or graphs or numerical strings that are currently used for that purpose are. The graphically rich images produced by this procedure are much too complex to facilitate comparison with any degree of speed. There will be no similar format nor shaping of the DNA data among any two separate individual DNAportraits taken from different individuals that would determine the art would be of the type DNA portrait.

### BRIEF SUMMARY OF THE INVENTION

A procedure to convert human DNA sequence data subsets to computer-generated art will allow visualization of a unique identifying sequence of DNA base-pairs based upon the Federal Bureau of Investigation open CODIS-compatible sample analysis methodology. The DNA sequence data that results from laboratory CODIS analysis is fed directly into a one-way proprietary hash algorithm, which in turn is fed into an algorithm which converts the numerical data into an image that is based upon standard fractal mathematical formulas. This in turn generates the visible image output. This image will be absolutely unique to the individual and will never be repeated unless the DNA sampled is either from an identical twin sibling or from a genetically cloned human being. Also, it will be the exact same image produced from a single individual's DNA sample no matter what point in time during the existence of the individual that the sample is taken. A DNA sample taken at a subject's birth will yield the same image when it is taken later in the subject's life or even after the subject's death if sampled from the subject's remains. The sole purpose of this new product will be to create art algorithmically based strictly on an individual subject's DNA sample. Its purpose is not designed nor intended to facilitate efficient, simple, and quick comparison of two different DNA samples as the standard charts or graphs or numerical strings that are currently used for that purpose are. The graphically rich images produced by this procedure are much too complex to facilitate comparison with any degree of speed.

### DETAILED DESCRIPTION

A graphical representation of human DNA is computer-generated with a high level of artistic quality. The artistic image is the result of processing a selection of STR loci from a DNA analysis using standard DNA sequencing procedures using either STR and/or RFLP procedures in the laboratory. This standard sequence is fed into several computer algorithms in series and analyzed to create a unique graphical fingerprint. This portrait is unique to the individual as is his actual physical fingerprint. Whether the DNA-sequencing/art procedure is done at birth, death, or any time in between the two, or even after death, the computer generated art image will be exactly identical and statistically unique. The only possible way of generating the same identical art-image would be to DNAportrait identical twins (born from the same fertilized egg cell) or to DNA portrait an individual and his genetically identical clone. The statistical probability of two individuals having identical portraits is approximately 1 in 30 billion. A summary of the procedure follows.

1. Using a buccal saliva swab, genetic material is sampled from an individual's inner cheek.

2. A CODIS-compatible sequence is done in the lab using a specific set of 13 STR loci.

These include, but are not limited to:

d18s51, d21s11, d3s1358, fga, csf1po, d18s539, d7s820, d8s1179, tpox.

This process yields a set of 26 three-digit integers. Additional loci may be taken from the individual's Y chromosome and mytochondrial DNA depending on the individual's sex and ethnicity to ensure uniqueness.

3. A standard MD5 hash is done on this data stream and the data is created as a numeric string of 32 characters that results from the hash functions. The MD5 hash is executed from between 1 and 62,990,927,829 times, until a condition is met in one of the following steps.

4. This sequence is fed into a fractal generation program using standard fractal mathematical algorithms, currently the program being used is ultrafractal, but any program that uses fractal math can be used. Currently the algorithms used are the Julia set and Mandelbrot set, but others may be used in the future. The formulas used include:

z(0)=pixel; z(n+1)=z(n)ˆ 2+c. With two parameters: real and imaginary parts of c.

z(0)=pixel; z(n+1)=z(n)ˆ 4+c. With two parameters: real and imaginary parts of c.

z(0)=c=pixel; z(n+1)=z(n)ˆ 2+c. With two parameters: real & imaginary perturbations of z(0)

The image is generated by recursively executing multiple times one or more of the above formulas until condition ARTFOUND is reached. See step 6 below.

5. This process results in an image containing up to 16 million colors and spanning 9,600 pixels in width and 12,000 pixels in height. This is a total of 115,200,000 individual color elements or pixels. Each pixel may be one of 16 million different colors.

6. Measurements are taken across the picture area (by computer) and steps 3 through 5 are repeated up to 62,990,927,829 times until the condition ARTFOUND is achieved ie. measurements yield greater than 30 unique colors with a coverage of the pixels greater than 3% of the 115,200,000 pixels, i.e. more than 3,456,000 pixels of each of the 30 colors identified. The change in each iteration is initiated by sequencing the 3-digit integer at the end of the data stream from step 2. These may also be layered to continue the recursion until this ARTFOUND condition is reached.

7. For a color DNAportrait, this image is then printed on a color computer printer or stored as a computer file in JPG or TIFF format.

What I claim as my invention is:

1. Human DNA samples create a unique work of art through the algorithms used in this procedure without having a similar format or shaping to the data amongst different DNA samples as a standard charting or graphical depiction currently provides. (ie. With a DNAportrait, there is no similarity between any two different DNA samples that can link the graphic image as being of the type DNAportrait when compared to standard methods used today such as a simple graphing, plotting, or charting of the data that would share a similarly arranged format for displaying the sequence data in itself labeling or signaling the fact that it is of the type “graphical chart of sequence data”.

2. The human DNA samples data will be displayed as artwork not merely as a sequence of numerical nor graphically charted data streams and this is the singular characteristic of this invention over the standard methods currently in place for displaying and comparing the uniqueness of DNA sequence data in a tabular or charted form.

3. Standard CODIS-compatible DNA sequence data streams (as used by the Federal Bureau of Investigation to uniquely identify an individual) are transformed from a series of numerical data into visible artwork that is much more highly complex in terms of color content, gradation and shading of color data by this procedure.
